(19) United States
(12) Patent Application Publication
(10) Pub. No.: US 2021/0402024 A1
(43) Pub. Date: Dec. 30, 2021

(54) UVC-LED light technology integrated for use in cell phone and tablets.

(71) Applicant: Roger Pawson, San Diego, CA (US)

(72) Inventor: Roger Pawson, San Diego, CA (US)

(21) Appl. No.: 17/349,062

(22) Filed: Jun. 16, 2021

Related U.S. Application Data
(60) Provisional application No. 63/046,261, filed on Jun. 30, 2020.

(51) Int. Cl. A61L 2/10; H04M 1/02; H04M 1/72466; A61L 2/26

(52) U.S. Cl. CPC A61L 2/10; H04M 1/026; A61L 2202/16; A61L 2/26; A61L 2202/11; H04M 1/72466

(57) ABSTRACT

A handheld device with a UCV-LED light incorporated into its body is taught. The light can be used to sanitize and disinfect an area in front of the user. The light is powered by the handheld device's battery and is controlled by the touchscreen of the device. The UCV-LED light both kills bacteria and disables viruses through the emission of UVC rays of light, with an optimal range of 256-284 nanometers. The light can be located next to the device's flashlight and is secured in a housing similar to that which secures a flashlight. It optionally has an indicator light that indicates the area to be sanitized and disinfected.

### CROSS REFERENCE TO RELATED APPLICATIONS

This application claims priority back to U.S. Provisional No. 63/046,261, with a filing date of Jun. 30, 2020, with a title of UVC-LED light technology integrated for use in cellphones & tablets, the contents of which are incorporated by reference into this utility patent application.

### STATEMENT REGARDING FEDERALLY SPONSORED RESEARCH OR DEVELOPMENT

This invention was not federally sponsored.

### BACKGROUND OF THE INVENTION

### Field of the Invention

This invention relates to the general field of sanitizing devices, and more specifically to a cell phone or tablet into which a sanitizing UVC-LED light has been incorporated. The device allows a user to sanitize an area by using a commonly-owned and commonly-carried device as opposed to have to bring along a specific sanitizing device to perform the same function.

### Brief Description of Invention

The invention is a handheld device with a UVC-LED light incorporated into its body. In a preferred embodiment, the light is located in a socket on the back of the device. The light can be used to sanitize and disinfect an area in front of the user and has an optional guide light to show what area is to the cleaned. The light is powered by the handheld device's battery and is controlled by the touchscreen of the device. The UVC-LED light both kills bacteria and disables viruses through the emission of UVC rays of light, with an optimal range of 256-284 nanometers. The light can be located next to the device's flashlight and is secured in a housing similar to that which secures a flashlight. The invention is particularly suitable for cell phones, but can also be used on tablets and any other device with a touchscreen that is carried on a person.

### Statement of the Problem

Sanitizing and disinfecting surfaces has long been a desired result in many facets of civilization. Since the recent Covid epidemic, however, the need for sanitary surfaces has become even more important. There exist a number of devices that can be used to sterilize and disinfect areas, but these area often large and bulky, or wet and have odors. Thus, there is a need for a device that does not take up additionally room in a person's purse, backpack or pocket, and yet provides superior sanitizing and disinfecting abilities.

The current invention provides just such a solution by having a UVC-LED light built into the body of a handheld device such as a cellphone, tablet, or personal digital assistant. The light can be located in a socket on the back of the device and controlled through the touchscreen, with power supplied by the device's battery. A guide light can show the area to be cleaned, and by simply pressing the “UVC-LED” icon on the screen, the person can activate the UVC-LED light and direct it to a specific area that he/she wants sanitized and disinfected. The UVC-LED light both kills bacteria and disables viruses through the emission of UVC rays of light, with an optimal range of 256-284 nanometers. In a preferred embodiment, the UVC-LED light is located next to the device's flashlight, and controlled in a similar manner.

### STATEMENT OF THE INVENTION

The invention is a device into which a UVC-LED light has been incorporated; ideally, on the back of the device.

The UVC-LED light is controlled by a touchscreen on the device into which it has been embedded and is powered by the device's battery.

The UVC-LED light has an optimum range of 256-284 nanometers.

A cellphone is an optimum device into which the UVC-LED light can be incorporated, but it can also be used in tablets, PDA's and other devices with a touchscreen.

There has thus been outlined, rather broadly, the more important features of the invention in order that the detailed description thereof may be better understood, and in order that the present contribution to the art may be better appreciated. There are additional features of the invention that will be described hereinafter and which will form the subject matter of the claims appended hereto. The features listed herein and other features, aspects and advantages of the present invention will become better understood with reference to the following description and appended claims. The accompanying drawings, which are incorporated in and constitute part of this specification, illustrate embodiments of the invention and, together with the description, serve to explain the principles of the invention.

It should be understood the while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

### BRIEF DESCRIPTION OF THE FIGURES

One preferred form of the invention will now be described with reference to the accompanying drawings.

FIG. 1 is a back view of a cell phone that has had its back removed to show the location of various components of the invention.

FIG. 2 is a side view of a cell phone illustrating how the invention does not affect the slim design of a cell phone.

FIG. 3 is a front view of the screen of a cell phone showing the location of the ON/OFF button.

FIG. 4 is a schematic view of the electrical components of the invention.

FIG. 5 is a back, perspective view of a cell phone that has incorporated the invention into its design.

FIG. 6 is a front view of a cell phone screen showing the UVC-LED light icon.

FIG. 7 is a front view of a cell phone screen showing the UVC-LED light icon.

FIG. 8 is a side, perspective view of the iWand embodiment of the invention with the back of the unit removed.

FIG. 9 a side, perspective view of the iWand embodiment of the invention with the back of the unit intact.

### DETAILED DESCRIPTION OF THE FIGURES

Many aspects of the invention can be better understood with references made to the drawings below. The components in the drawings are not necessarily drawn to scale. Instead, emphasis is placed upon clearly illustrating the components of the present invention. Moreover, like reference numerals designate corresponding parts through the several views in the drawings. Before explaining at least one embodiment of the invention, it is to be understood that the embodiments of the invention are not limited in their application to the details of construction and to the arrangement of the components set forth in the following description or illustrated in the drawings. The embodiments of the invention are capable of being practiced and carried out in various ways. In addition, the phraseology and terminology employed herein are for the purpose of description and should not be regarded as limiting.

It should be understood that while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

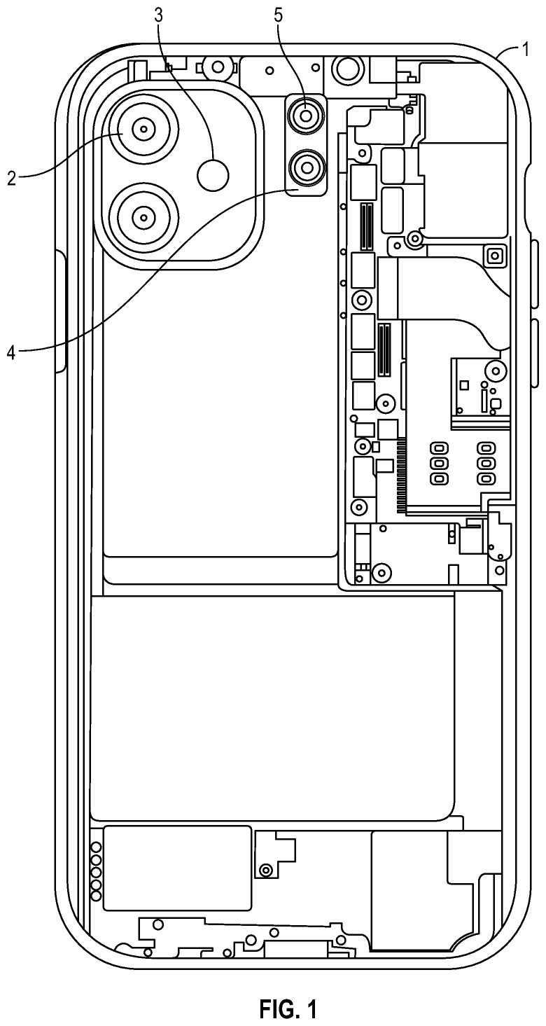

FIG. 1 is a back view of a cell phone that has had its back removed to show the location of various components of the invention. In this figure, the UVC-LED light has been incorporated into a cell phone. The cell phone 1 has a camera 2, a flashlight 3, a UVC-LED sterilizer lens 4 (behind where there is a UVC-LED light, not shown in this figure) and a sterilizing area indicator light 5. The sterilizing area indicator light 5 illuminates the area that will be sanitized when the user turned on the UVC-LED light 4.

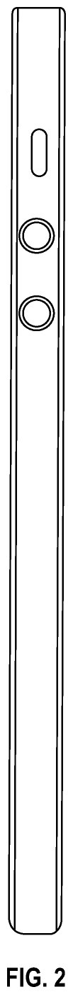

FIG. 2 is a side view of a cell phone illustrating how the invention does not affect the slim design of a cell phone. Both the indicator light (#5 in FIG. 1) and the sterilizer lens (#4 in FIG. 1) are mounted flush with the back case of the cell phone, so that they do not protrude from the back of the cell phone or add width to it.

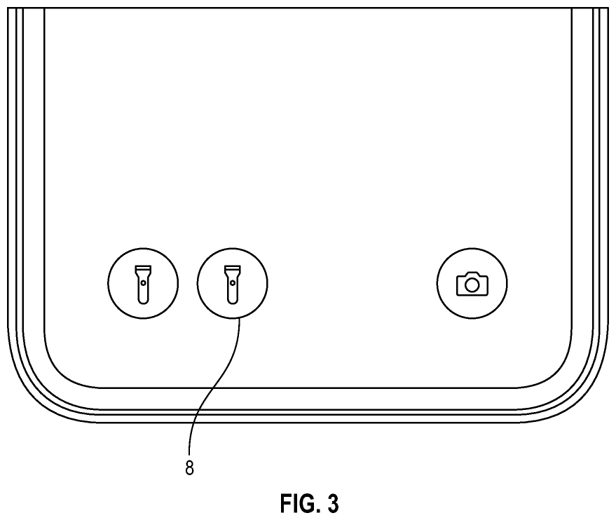

FIG. 3 is a front view of the screen of a cell phone showing the location of the ON/OFF button. A user can easily turn the sanitizing feature on by merely pressing this icon, very similar to how a user activates the Flashlight feature of a cell phone.

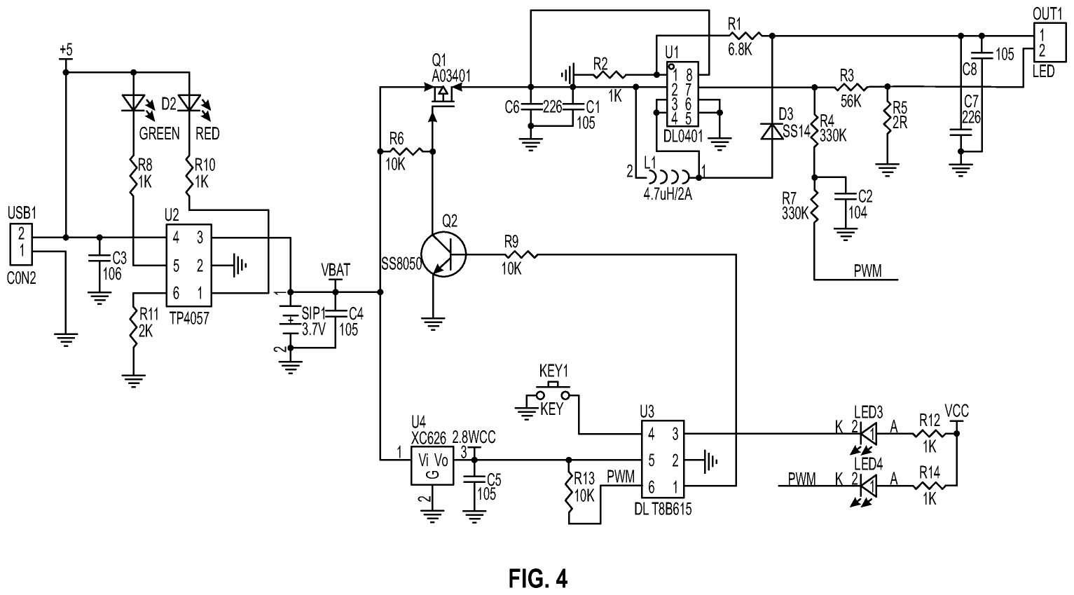

FIG. 4 is a schematic view of the electrical components of the invention.

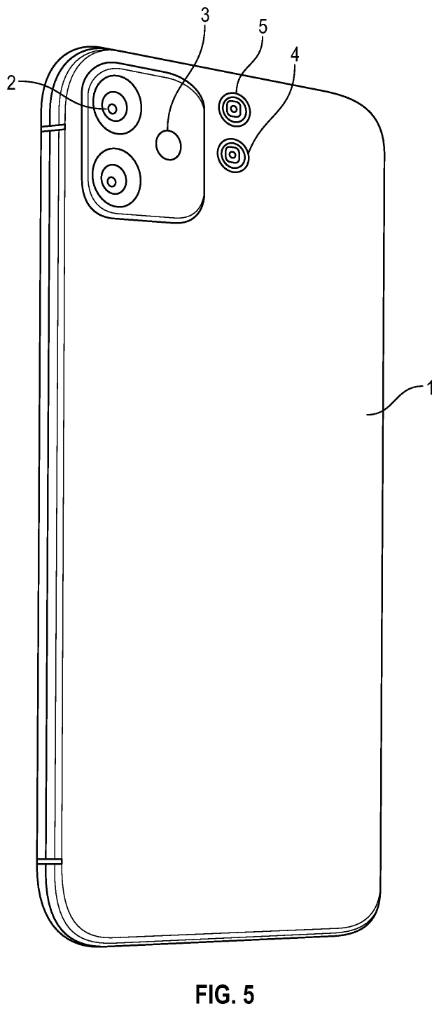

FIG. 5 is a back, perspective view of a cell phone that has incorporated the invention into its design. The cell phone 1 has a traditional back case with a camera 2 and a flashlight 3, but with two additional holes/mounts built into it: one for the indicator light 5 and one for the sanitizer 4. Upon activating the indicator light 5, an indicator light illuminates the area that will be sanitized should the sanitizer 4 be activated. This way, a user can preview the area to be sanitized before actually beginning the sanitizing process.

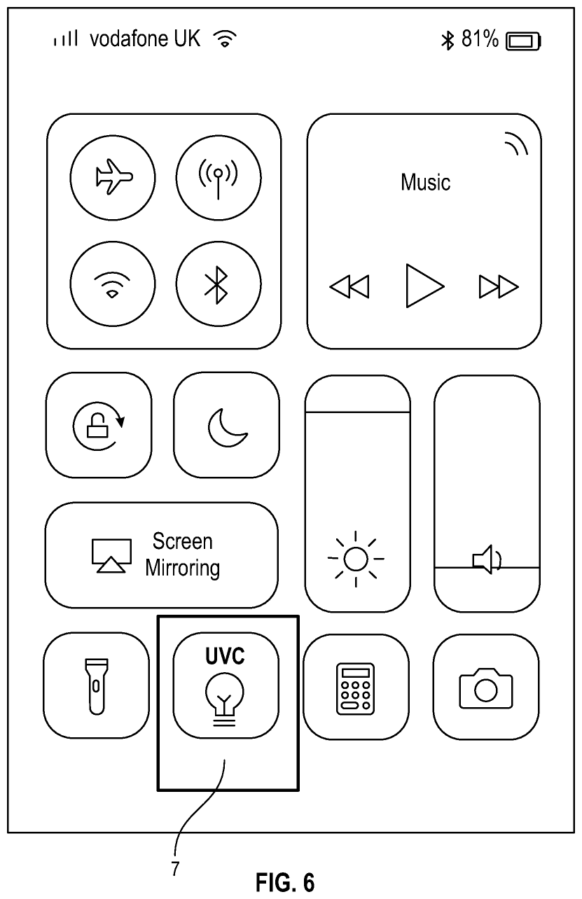

FIG. 6 is a front view of a cell phone screen showing the UVC-LED light icon. FIG. 2 is a front view of a proposed touchscreen control icon for turning on and off the UVC-LED light. On the touchscreen 6 are a number of icons, including the UVC-LED light icon 7. To activate the UVC-LED light icon 7, users press this icon with a finger.

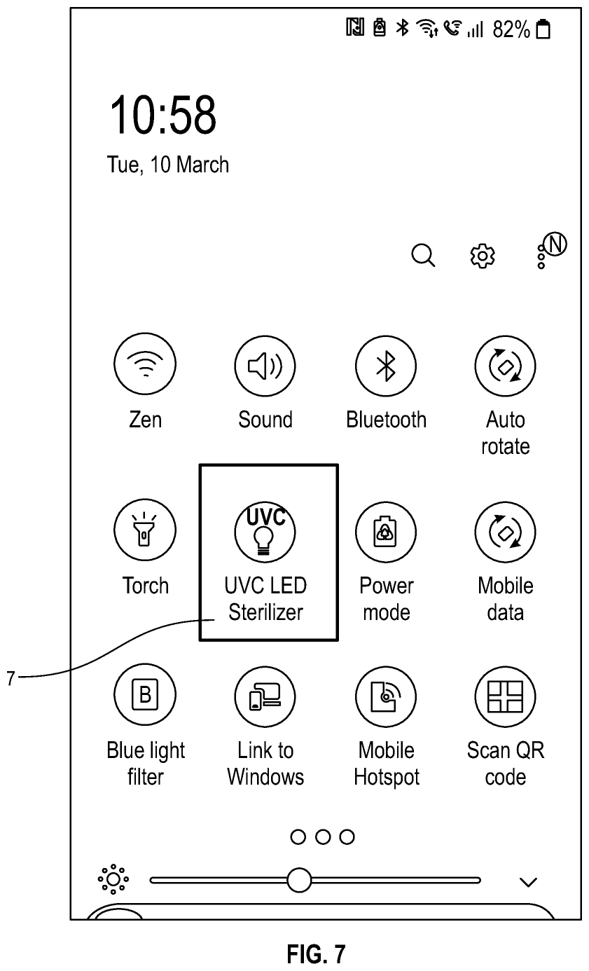

FIG. 7 is a front view of a cell phone screen showing the UVC-LED light icon.

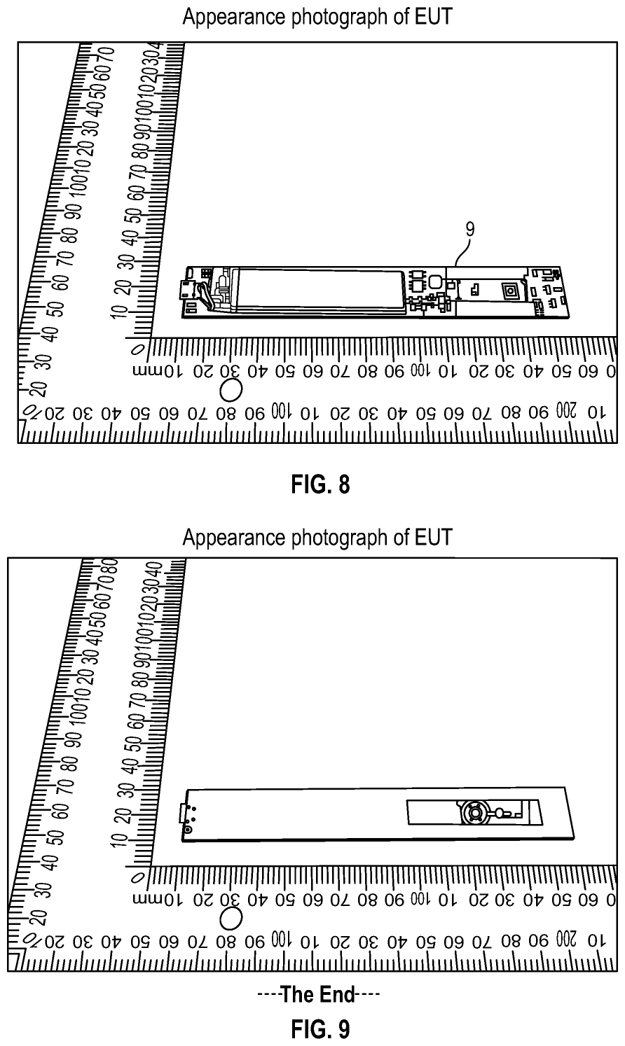

FIG. 8 is a side, perspective view of the iWand embodiment of the invention with the back of the unit removed.

FIG. 9 a side, perspective view of the iWand embodiment of the invention with the back of the unit intact.

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

What I claim is:

1. A sanitizing device, consisting of: a UVC-LED light, housing, a UVC-LED lens, a UVC-LED indicator light, a source of power and a control chip, where, the housing removably attaches the UVC-LED light to a shell of a touchscreen device, where the UVC-LED lens focuses an amount of light from the UVC-LED light, where the source of power powers the UVC-LED light, where the control chip takes a first signal from a touchscreen icon and allows power from the source of power to turn on the UVC-LED light, and where the control chip takes a second signal from a touchscreen icon and cuts off power from the source of power to the UVC-LED light, where the source of power is a cell phone battery.

2. The device of claim 1, where the UVC-LED Light has a range, and that range is between 200-300 nanometers

3. The device of claim 2, where the range is between 256-284 nanometers.

4. The device of claim 3, additionally comprising an indicator light.

5. The device of claim 4, where there is a touchscreen indicator light icon by which a user can activate the indicator light, where the indicator light illuminates a proposed sanitization area, where the proposed sanitization area is approximately a sanitization zone, where the sanitization zone is a region to be sanitized by the UVC-LED light.

6. The device of claim 5, where the housing is a cell phone housing and where the housing is a cell phone housing and where a plurality of electrical wires connect the battery to the indicator light, the battery to the UVC-LED light, and connect a plurality of desktop icons to the indicator light and to the UVC-LED light.

7. The device of claim 5, where the housing is a tablet housing.

8. A sanitizing device, comprising: a UVC-LED light, housing, a UVC-LED lens, a UVC-LED indicator light, a source of power and a control chip, where, the housing removably attaches the UVC-LED light to a shell of a touchscreen device, where the UVC-LED lens focuses an amount of light from the UVC-LED light, where the source of power powers the UVC-LED light, where the control chip takes a first signal from a touchscreen icon and allows power from the source of power to turn on the UVC-LED light, and where the control chip takes a second signal from a touchscreen icon and cuts off power from the source of power to the UVC-LED light.

9. The device of claim 8, where the UVC-LED Light has a range, and that range is between 200-300 nanometers

10. The device of claim 9, where the range is between 256-284 nanometers.

11. The device of claim 8, where there is a touchscreen indicator light icon by which a user can activate the indicator light, where the indicator light illuminates a proposed sanitization area, where the proposed sanitization area is approximately a sanitization zone, where the sanitization zone is a region to be sanitized by the UVC-LED light.

12. The device of claim 8, where there is a touchscreen indicator light icon by which a user can activate the indicator light, where the indicator light illuminates a proposed sanitization area, where the proposed sanitization area is approximately a sanitization zone, where the sanitization zone is a region to be sanitized by the UVC-LED light, and, where the range is between 256-284 nanometers.

13. The device of claim 8, where the source of power is a cell phone battery, where there is a touchscreen indicator light icon by which a user can activate the indicator light, where the indicator light illuminates a proposed sanitization area, where the proposed sanitization area is approximately a sanitization zone, where the sanitization zone is a region to be sanitized by the UVC-LED light, where there is a touchscreen indicator light icon by which a user can activate the indicator light, where the indicator light illuminates a proposed sanitization area, where the proposed sanitization area is approximately a sanitization zone, where the sanitization zone is a region to be sanitized by the UVC-LED light, and, where the range is between 256-284 nanometers.

14. The device of claim 13, where the housing is a cell phone housing.

15. The device of claim 13, where the housing is a tablet housing.

16. The device of claim 13, where the housing is selected from the group consisting of a cell phone housing, tablet housing, and a personal digital assistant housing.

17. A sanitizing device, comprising: a UVC-LED light, housing, a UVC-LED lens, a UVC-LED indicator light, a source of power and a control chip, where, the housing removably attaches the UVC-LED light to a shell of a touchscreen device, where the UVC-LED lens focuses an amount of light from the UVC-LED light, where the source of power powers the UVC-LED light, where the control chip takes a first signal from a touchscreen icon and allows power from the source of power to turn on the UVC-LED light, and where the control chip takes a second signal from a touchscreen icon and cuts off power from the source of power to the UVC-LED light, where the UVC-LED Light has a range, and that range is between 200-300 nanometers, where there is a touchscreen indicator light icon by which a user can activate the indicator light, where the indicator light illuminates a proposed sanitization area, where the proposed sanitization area is approximately a sanitization zone, where the sanitization zone is a region to be sanitized by the UVC-LED light, where there is a touchscreen indicator light icon by which a user can activate the indicator light, where the indicator light illuminates a proposed sanitization area, where the proposed sanitization area is approximately a sanitization zone, where the sanitization zone is a region to be sanitized by the UVC-LED light.

18. The device of claim 17, where the range is between 256-284 nanometers, and where the source of power is a rechargeable battery.

19. The device of claim 18, where the housing is selected from the group consisting of a cell phone housing, tablet housing, and a personal digital assistant housing.

20. The device of claim 18, where the housing is a cell phone housing and where a plurality of electrical wires connect the battery to the indicator light, the battery to the UVC-LED light, and connect a plurality of desktop icons to the indicator light and to the UVC-LED light.
